(19) United States
(12) Patent Application Publication
(10) Pub. No.: US 2004/0028646 A1
(43) Pub. Date: Feb. 12, 2004

(54) Particle formation

(76) Inventors: Remy Gross, Huntsville, AL (US); Andreas Kordikowski, Skipton (GB); Raymond Sloan, Bradford (GB)

Correspondence Address: NEKTAR THERAPEUTICS, 150 INDUSTRIAL ROAD, SAN CARLOS, CA, 94070, US

(21) Appl. No.: 10/622,094

(22) Filed: Jul. 17, 2003

(30) Foreign Application Priority Data
Jul. 19, 2002 (GB) 0216780.7

(51) Int. Cl. A61K 31/765

(52) U.S. Cl. 424/78.38

(57) ABSTRACT

A polyalkylene glycol (PAG)—preferably a polyethylene glycol—or a derivative or conjugate thereof, in particulate form, having a residual solvent content of 200 ppm or less and a volume mean particle diameter of 25 μm or less. Also methods for forming particulate products containing PAGs or derivatives or conjugates thereof, in particular by carrying out a GAS process—preferably a Nektar™ SCF process—on a solution or suspension of a target substance but using as the anti-solvent fluid a compressed fluid which at the point of its contact with the solution or suspension is at a temperature of 25° C. or below.

### FIELD OF THE INVENTION

The present invention relates to methods for forming particles of target substances and to particulate products of the methods.

### BACKGROUND TO THE INVENTION

Polyalkylene glycols, in particular polyethylene glycol (PEG, also known as polyethylene oxide (PEO) or polyoxyethylene (POE) or polyether glycol) are widely used as excipients in pharmaceutical formulations since they can help to enhance the water solubility, and hence bioavailability on administration, of pharmaceutically active materials. PEG in particular has low toxicity and immunogenicity and is particularly well known as an excipient for use with proteinaceous actives, for which it can help to improve shelf stability and also increase the half-life in vivo.

Polyalkylene glycols such as PEGs can be coformulated with active substances for instance as physical mixtures or as intimate solid dispersions. It is also possible, however, to conjugate a polyalkylene glycol (hereafter PAG) covalently to an active substance. This is achieved by “activating” the PAG, typically by replacing a terminal hydroxy group with a reactive functional group suitable for conjugation to the relevant active substance. The preparation of such “activated” PAGs, and their conjugation to active substances, are disclosed for example in EP-1 176 160, U.S. Pat. No. 6,214,966, U.S. Pat. No. 6,258,351, WO-99/45964, WO-01/00246, WO-01/24831, WO-01/26692, WO-01/45796, WO-01/46291, WO-01/47562, WO-01/62299 and WO-01/62827, with particular emphasis on PEGs.

Using such technology, significant difficulties can be experienced in preparing and in particular isolating the activated PAG product. Typically it is precipitated, following its synthesis, from an organic solvent such as dichloromethane, following which solvent evaporation and drying steps are required in order to achieve a particulate solid product. This can be a lengthy process; the precipitate may take a long time to settle following centrifugation and often long drying periods (for instance 2 to 4 days' air or oven drying) are needed. The result may be a coarse powder or flakes instead of the fast dissolving, fine particulate which would be more desirable for future processing. The product may also contain undesirably high levels of residual solvent, and the process itself requires high levels of organic solvents which must subsequently be disposed of. Yields, furthermore, can be low.

It would be preferable if activated PAGs could be more easily converted into a pure, dry particulate product, or still more preferably be prepared directly in particulate form.

Active substances such as proteins, peptides and small molecule pharmaceuticals may be conjugated with such activated PAGs to enhance their aqueous solubility, extend their circulating half life and improve their bioavailability and/or stability especially at high concentrations. This is of particular use in preparing injectable drug formulations. However, the resultant conjugate tends to be synthesised in aqueous solution, and if stored in this form lacks long-term stability, typically needing to be refrigerated and having a shelf life of only about 30 days. When prepared in particulate form (for instance by solvent extraction and drying), again poor handling properties and high residual solvent levels can result, yields are often low and processing lengthy. Again it would be preferable if the conjugates could be prepared in, or converted into, a particulate form with improved physicochemical properties, in particular with improved purity, which could be stored dry and re-hydrated, without undue loss of activity, prior to use.

A number of techniques are known for forming particles of a substance of interest (a “target substance”). Amongst them are processes which make use of supercritical or near-critical fluids as anti-solvents. This technique is generally known as GAS (Gas Anti-Solvent) and involves contacting a solution or suspension of the target substance, in a suitable fluid vehicle, with a supercritical or near-critical fluid (SCF/NCF) which is a nonsolvent for the substance but is miscible with the vehicle. The SCF/NCF, referred to in this context as an “anti-solvent”, extracts the vehicle and thus causes precipitation of the target substance.

A supercritical fluid is a fluid which is simultaneously above both its critical temperature T_{c} and its critical pressure P_{c}. “Near-critical fluid” is here used to refer to a fluid which is either (a) above its T_{c} but slightly below its P_{c}, (b) above its P_{c} but slightly below its T_{c} or (c) slightly below both its T_{c} and its P_{c}.

The basic GAS technique is described in Gallagher et al, “Supercritical Fluid Science and Technology”, ACS Symp. Ser., 406, p334 (1989) and is exemplified for instance in WO-90/03782. Other versions of technique are known, typically involving different ways of contacting the reagent fluids. These include “ASES” (Aerosol Solvent Extraction System—see U.S. Pat. No. 5,043,280), PCA (Precipitation using Compressed fluid Anti-solvent—see eg, Dixon et al, AIChE Journal, 1993, 26, 127), SAS (Solvent Anti-Solvent—see eg, Yeo et al, Biotechnology and Bioengineering, 1993, 41, 341) and the Nektar™ SCF particle formation process, formerly known as SEDS™ (Solution Enhanced Dispersion by Supercritical fluid—see WO-95/01221, WO-96/00610, WO-98/36825, WO-99/44733, WO-99/59710, WO-01/03821, WO-01/15664, WO-02/38127 WO-03/008082, and co-pending UK patent applications nos. 0300338.1 and 0300339.9).

Such techniques have enabled the formation of particulate products with desirable physicochemical characteristics (in particular particle size and size uniformity), often with a high degree of control over aspects such as the purity, morphology and yield of the product. This has seen wide application in the production of pharmaceutically active substances and their excipients.

There are considerable advantages to using SCF/NCF anti-solvents, in particular the wide variations in their solvent power which can be achieved by relatively small adjustments in their temperature and pressure. The most commonly used SCF/NCF is CO₂, which is non-toxic, non-flammable, generally inert and also relatively inexpensive. It also has a relatively low T_{c} (31° C./304 K) and P_{c} (74 bar).

Nevertheless, not all substances are suitable for processing using supercritical or near-critical fluids. Some are unable to withstand the temperatures needed in order to maintain the anti-solvent fluid in its supercritical or near-critical state. This may be the case, for instance, with certain biologically active materials such as proteins, peptides, nucleic acids and their derivatives. It is also the case for certain polymers which “melt”, or form glasses, at the required operating temperatures. This latter problem can be exacerbated if the anti-solvent itself (as in the case of CO₂ for instance) acts to lower the glass transition temperature of the polymer.

PAGs such as PEGs and polypropylene glycols are examples of such polymers. Dependent on their molecular weight, PEGs for instance tend to melt at temperatures between about 30 and 45° C., sometimes even below room temperature, and are therefore unsuitable for processing with supercritical or even near-critical CO₂.

### STATEMENTS OF THE INVENTION

It has surprisingly been found, despite the above, that PAGs (in particular PEGs) and their derivatives, and formulations containing them, may be processed by modifying the basic GAS technique, in order to obtain the above described advantages of the GAS process despite the apparent incompatibility of these polymers with supercritical fluid processing. This may in turn help to alleviate the long-felt problems associated with preparing such products by more conventional techniques.

According to a first aspect of the present invention there is provided a method for forming particles of a target substance which comprises a polyalkylene glycol (PAG) or a derivative or conjugate thereof, which method comprises carrying out a GAS process, preferably a Nektar™ SCF (SEDS™) process, on a solution or suspension of the target substance in a fluid vehicle (the “target solution/suspension”), but using as the anti-solvent fluid a compressed fluid which at the point of its contact with the target solution/suspension is at a temperature of 25° C. (298 K) or below.

The target substance may in particular comprise a polyethylene glycol (PEG), a polypropylene glycol (PPG), a copolymer of the two, a derivative or conjugate of any of these or a mixture of any of them. More particularly it comprises a PEG or a derivative or conjugate thereof.

Carrying out a GAS process on the target solution/suspension involves contacting it with a compressed fluid anti-solvent under conditions which allow the anti-solvent to extract the vehicle from the target solution/suspension and to cause particles of the target substance to precipitate from it. The conditions should be such that the fluid mixture thus formed between the anti-solvent and the extracted vehicle is still in a compressed state. The anti-solvent fluid should be a nonsolvent for the target substance and be miscible with the fluid vehicle, under the operating conditions at the point of particle formation.

Carrying out a Nektar™ SCF or SEDS™ process involves carrying out the above described GAS process, but using the anti-solvent fluid simultaneously both to extract the vehicle from, and to disperse, the target solution/suspension. In other words, the fluids are contacted with one another in such a manner that the mechanical (kinetic) energy of the anti-solvent can act to disperse the target solution/suspension at the same time as the anti-solvent mixes with and extracts the vehicle. “Disperse” in this context refers generally to the transfer of kinetic energy from one fluid to another, usually implying the formation of droplets, or of other analogous fluid elements, of the fluid to which the kinetic energy is transferred.

Suitable Nektar™ SCF processes are described in WO-95/01221, WO-96/00610, WO-98/36825, WO-99/44733 and WO-99/59710, WO-01/03821, WO-01/15664, WO-02/38127 and WO-03/008082, and in co-pending UK patent applications nos. 0300338.1 and 0300339.9; these may be used in the method of the present invention provided the anti-solvent is a compressed fluid at a temperature of 25° C. or below. In particular, the target solution/suspension and the anti-solvent are preferably contacted with one another in the manner described in WO-95/01221 and/or WO-96/00610, being co-introduced into a particle formation vessel using a fluid inlet means which allows the mechanical energy (typically the shearing action) of the anti-solvent flow to facilitate intimate mixing and dispersion of the fluids at the point where they meet. The target solution/suspension and the anti-solvent preferably meet and enter the particle formation vessel at substantially the same point, for instance via separate passages of a multi-passage coaxial nozzle—such a nozzle suitably has a convergent tip and one or more of its inner passages ideally terminates a small distance upstream (in the direction of fluid flow in use) of the outlet of the outer passage, thus allowing a small degree of internal mixing between fluids introduced through the respective passages, before they exit the nozzle.

Alternatively, it may be preferred for the target solution/suspension and the anti-solvent to be contacted with one another in the manner described in WO-03/008082, or in co-pending UK patent applications nos. 0300338.1 or 0300339.9. Here the two fluids enter a particle formation vessel at separate, although close, locations, again preferably in such a way that the mechanical energy of the anti-solvent assists intimate mixing and dispersion of the fluids at their point of contact. In such cases, the anti-solvent velocity as it enters the particle formation vessel is preferably near-sonic, sonic or supersonic. The anti-solvent is suitably introduced via a nozzle, again conveniently having a convergent tip.

References in this specification to a fluid entering a vessel are to the fluid exiting an inlet means (for example, a nozzle) used to introduce the fluid into the vessel. For these purposes, therefore, the inlet means is to be considered as upstream of the vessel in the direction of fluid flow, although parts of it (in particular its outlet) may be located physically within the vessel.

Other suitable SEDS™ processes are disclosed in WO-99/52507, WO-99/52550, WO-00/30612, WO-00/30613, WO-00/67892 and WO-02/058674. Such documents relating to Nektar™ SCF or SEDS™ processes, and those listed above, are intended to be read together with the present application. Moreover in the context of the present invention, a Nektar™ SCF process may be a combination of two or more of those described in the above listed documents.

In the present context, references to an anti-solvent fluid being in a compressed state mean that, at the relevant operating temperature, it is above its vapour pressure, preferably above atmospheric pressure, more preferably from 70 to 200 bar or from 80 to 150 bar. The anti-solvent fluid is preferably a fluid which is a gas at atmospheric pressure and ambient temperature. In other words, it should have a vapour pressure above 1 bar at ambient temperature (eg, at 18 to 25° C., such as at 22° C.).

More preferably “compressed” means close to, at or yet more preferably above the critical pressure P_{c} for the fluid or fluid mixture concerned. The anti-solvent may be a supercritical or near-critical fluid, although it may alternatively be a compressed liquid such as for instance liquid CO₂. In practice, the pressure is likely to be in the range (1.01-9.0)P_{c}, preferably (1.01-7.0)P_{c} for a supercritical or near-critical fluid anti-solvent, or for example (0.7-3.0)P_{c}, preferably (0.7-1.7)P_{c}, for a compressed liquid anti-solvent such as liquid CO₂.

As used herein, the term “supercritical fluid” means a fluid at or above its critical pressure (P_{c}) and critical temperature (T_{c}) simultaneously. In practice, the pressure of such a fluid is likely to be in the range (1.01-9.0)P_{c}, preferably (1.01-7.0)P_{c}, and its temperature in the range (1.01-4.0)T_{c} (measured in Kelvin). However, some fluids (eg, helium and neon) have particularly low critical pressures and temperatures, and may need to be used under operating conditions well in excess of (such as up to 200 times) those critical values.

“Near-critical fluid” is here used to refer to a fluid which is either (a) above its T_{c} but slightly below its P_{c}, (b) above its P_{c} but slightly below its T_{c} or (c) slightly below both its T_{c} and its P_{c}. The term “near-critical fluid” thus encompasses both high pressure liquids, which are fluids at or above their critical pressure but below (although preferably close to) their critical temperature, and dense vapours, which are fluids at or above their critical temperature but below (although preferably close to) their critical pressure.

By way of example, a high pressure liquid might have a pressure between about 1.01 and 9 times its P_{c}, and a temperature from about 0.5 to 0.99 times its T_{c}. A dense vapour might, correspondingly, have a pressure from about 0.5 to 0.99 times its P_{c}, and a temperature from about 1.01 to 4 times its T_{c}.

The terms “compressed fluid”, “supercritical fluid” and “near-critical fluid” each encompass a mixture of fluid types, so long as the overall mixture is in the compressed, supercritical or near-critical state respectively.

“PAG or PAG derivative” for the purposes of this invention includes any polymeric molecule containing in its polymeric backbone the alkylene glycol repeat unit (O(CH₂)ₙ), where n is an integer from 2 to 8, preferably 2 or 3 (ethylene glycol or propylene glycol), more preferably 2. Such a molecule preferably contains (O(CH₂)ₙ)ₘ where m is an integer from 40 to 3,000, typically from 100 to 2,000, more typically from 100 to 1,000 or from 200 to 1,000. The term “PAG or PAG derivative” includes any polymeric molecule in which at least 50%, preferably at least 70%, more preferably at least 80% or at least 90%, of the repeat units (regardless of the positions of those repeat units, and therefore of the structure (eg, linear or branched) of the polymer) are alkylene glycol units, preferably units of the formula (O(CH₂)ₙ) where n is as defined above. The term therefore encompasses copolymers and terpolymers (in each case either random or block) containing (i) two or more different alkylene glycol repeat units and/or (ii) other repeat units in addition to the alkylene glycol units.

A PAG chain is therefore any polymeric chain containing the alkylene glycol repeat unit, as described above. Again it may contain two or more different types of alkylene glycol unit, and/or other monomer units, however preferably at least 50%, more preferably at least 70%, most preferably at least 80% or 90% of its repeat units are alkylene glycol units.

PAG as used herein is meant to encompass (i) linear PAGs (both monofunctional and difunctional or “dumbell”-like), (ii) branched PAGs having more than one PAG chain extending from a central core structure, for instance those having the general formula T-(PAG)ₚ where PAG is a PAG chain, T is a multivalent “core” group for example a residue of a polyol such as pentaerythritol, lysine, sorbitol or a glycerol, and p represents the number of arms and is typically from 2 to 120, ideally at least 5, (iii) pendant PAGs having reactive functional groups (for instance carboxyl groups) along the polymer backbone as well as or instead of at their terminae, (iv) forked PAGs represented by the formula PAG-(V)ᵣ where V is a group suitable for attachment to another moiety and r is an integer of 2 or greater, and in which the group V may be attached to two or more other groups such as PAG chains, (v) dendritic PAGs having highly branched structures, and the like. The term “PAG derivative” may be construed accordingly. Branched PAGs and their derivatives may for instance be prepared by addition of an alkylene oxide to various polyols, such as glycerol, pentaerythritol and sorbitol, the polyol representing the multivalent “core”.

A PAG chain may itself be linear, branched, pendant, forked or dendritic.

Where a PAG or PAG derivative includes propylene glycol or higher alkylene glycol repeat units, and the repeat units have a branched structure, its conformation may be isotactic, syndiotactic or atactic. Isotactic polymers tend to have relatively high levels of crystallinity and melting points, syndiotactic polymers intermediate melting points and partial crystallinity and atactic polymers relatively low melting points and amorphous natures. The present invention may thus be of particular use in processing syndiotactic and especially atactic polymers.

A PAG or PAG derivative may be cross-linked, for instance as described in U.S. Pat. No. 6,258,351 or WO-01/00246.

“PAG derivative” is meant to encompass polymers having any of a number of suitable functional groups attached to the polymeric backbone or more preferably at its terminae, depending upon its structure and intended use. A PAG derivative may therefore be monofunctional (ie, having one reactive functional group (or precursor thereof) suitable, eg, for attachment to a drug moiety, and one inert or relatively unreactive terminus such as methyl), bifunctional (having two reactive functional groups or precursors thereof for reaction with two other moieties, wherein the functional groups may be the same or different), multifunctional, etc. Additionally, a PAG derivative may contain one or more spacer or linker groups (these terms may by used interchangeably) separating the main polymer portion of the molecule from a reactive terminus. PAGs and PAG derivatives may also contain one or more hydrolysable functionalities within the polymer chain for in-vivo hydrolysis of portions of the polymer chain.

According to the first aspect of the invention, the target substance is typically either

(a) a polyalkylene glycol (PAG) of the general formula (I):

R¹—(O(CH₂)ₙ)ₘ—O—R² (I)

where n and m are integers as defined above, and R¹ and R² are each independently either hydrogen or an unreactive end group such as alkyl, in particular methyl or ethyl; or

(b) a PAG derivative, as defined above, which contains alkylene glycol repeat units and more preferably contains the group (O(CH₂)ₙ)ₘ where n and m are as defined above, together with one or more functional groups on the main polymer chain(s) and/or at its terminae; or

(c) a PAG conjugate, which comprises a PAG derivative as defined above, covalently bound to one or more active substances.

The PAG, PAG derivative or PAG conjugate is preferably hydrophilic.

The PAG derivative may be an “activated PAG” containing one or more reactive functional groups X on the main polymer chain(s) and/or at its terminae, where X is an “activating” group selected to facilitate subsequent conjugation of the polymer to an active substance such as a protein or peptide. Examples of such activated PAGs are well known and are disclosed, for instance, in EP-1 176 160, U.S. Pat. No. 6,214,966, U.S. Pat. No. 6,258,351, WO-99/45964, WO-01/00246, WO-01/24831, WO-01/26692, WO-01/45796, WO-01/46291, WO-01/47562, WO-01/62299 and WO-01/62827.

In particular an activated PAG may have the general formula (II):

R¹—(O(CH₂)ₙ)ₘ—X (II),

where R¹, X, n and m are as defined above.

An activating group X comprises a functional group which is able to react with a group on another molecule such as a drug, targeting moiety or the like. It is preferably “active” in the sense that it reacts readily with electrophilic or nucleophilic groups on other molecules, without the need for strong catalysts or highly impractical reaction conditions—for example, an “active” ester might react readily with nucleophilic groups such as amines, typically reacting in a matter of minutes in an aqueous medium without the need for catalysts.

Suitable functional groups include (a) sulphones, in particular vinyl sulphones such as SO₂—CH═CH₂ and active alkyl (in particular ethyl) sulphones such as SO₂—CH₂—CH₂—Lₑ where Lₑ is a leaving group such as halide, in particular chloride; (b) active esters, such as carboxylic acid esters, carbonate esters, sulphonate esters, isocyanates, isothiocyanates, acrylates, methacrylates, benzotriazolyl esters or succinimidyl (preferably carboxylate or carbonate) esters, examples being N-hydroxysuccinimidyl esters or N-sulphosuccinimidyl esters; (c) acetals, diones, aldehydes and aldehyde hydrates; (d) epoxides; (e) amines, in particular NH₂; (f) alcohols and thiols; (g) maleimides, for instance N-maleimides as described in WO-01/62827; (h) hydrazides; (i) dithiopyridines and vinyl pyridines; (j) iodoacetamides; (k) carbamate (in particular carbamide) precursors of the general formula —O—C(O)—X′, where X′ is a reactive group such as halide, hydroxyl, 1-benzotriazolyloxy, p-nitrophenyloxy, 1-imidazolyl, N-sulphosuccinimidyloxy or especially N-succinimidyloxy, preferred versions being of the form —Ar—O—C(O)—X where Ar is an aromatic group; (l) carboxylic acids; (m) mesylates, tosylates or tresylates; (n) alkenyls; (o) acrylamides; (p) glyoxals; (q) halides; and (r) alkoxy groups or hydroxyl.

Suitable “activated PEGs” of this type are described in the 2001 catalogue of Shearwater Corporation (Huntsville, Ala., US) entitled “Polyethylene Glycol and Derivatives for Biomedical Applications”, and in later such catalogues available from the same company. Preferred groups X are active esters such as N-hydroxysuccinimidyl (NHS) esters, aldehydes and aldehyde precursors such as acetals, maleimides, benzotriazole carbonate and carboxyl.

The activated PAG may include a linking group Lᵢ by which the reactive group X is bound to the rest of the polymer. Generally Lᵢ may be either hydrolytically stable or unstable, depending on the intended use of the derivative. Suitable hydrolytically unstable (ie, hydrolysable) linking groups can include those containing carboxylate esters, phosphate esters, orthoesters, anhydrides, acetals, ketals, imines, ester-linked amino acids, peptides or oligonucleotides, for instance as described in U.S. Pat. No. 6,214,966 or U.S. Pat. No. 6,258,351. Hydrolytically stable linking groups can include groups containing optionally substituted alkylene or alkenylene (preferably C₁—C₆) groups (that may be branched or straight-chain), ethers, thioethers, amines, amides, imides, carbamides and certain esters. Photolytically degradable linking groups include cinnamate dimers and cinnamylidine dimers. The linking group may carry substituents (for instance, sterically hindering groups) to influence its stability.

The linking group may itself contain active moieties, for example amino groups, through which the Lᵢ-polymer attachment may be formed.

The group X may be multi-functional, ie, reactive at two or more sites. The linking group Lᵢ, if present, may be or comprise a multivalent group (for instance CH, the carbon atom of which may form attachments to 1, 2 or 3 other atoms depending on its degree of saturation) and be linked to more than one reactive group X and/or to more than one group R¹—(O(CH₂)ₙ)ₘ. A further link (for example a C₁-C₆ alkylene or alkenylene group) may be present between each group X and the multivalent group Lᵢ.

The group X may therefore be linked to more than one PAG chain, eg, to more than one group of formula R¹—(O(CH₂)ₙ)ₘ.

“Activated” PAG derivatives containing a terminal group X may for example be synthesised as described in EP-1 176 160, U.S. Pat. No. 6,214,966, U.S. Pat. No. 6,258,351, WO-99/45964, WO-01/00246, WO-01/24831, WO-01/26692, WO-01/45796, WO-01/46291, WO-01/47562, WO-01/62299 and/or WO-01/62827, by appropriate reactions at the terminal —OH group of a PAG base polymer.

The term “PAG derivative” additionally encompasses salts, solvates, esters, analogues and other derivatives (including precursors) of PAGs and activated PAGs as described above, in particular pharmaceutically acceptable forms.

In accordance with the present invention, a PAG conjugate is an “activated PAG” of the type described above, which has been conjugated by means of a covalent bond to an active substance. It is typically a PAG-active substance conjugate of the general formula (III):

R¹—(O(CH₂)ₙ)ₘ—Y—Z (III),

where Z is an active substance for instance as referred to in EP-1 176 160, U.S. Pat. No. 6,214,966, U.S. Pat. No. 6,258,351, WO-99/45964, WO-01/00246, WO-01/24831, WO-01/26692, WO-01/45796, WO-01/46291, WO-01/47562, WO-01/62299 and/or WO-01/62827, and Y represents a covalent link formed between an activating group X (optionally with a linking group Lᵢ) as defined above and a reactive group in the active substance. The active substance may in particular be a biologically active substance, more particularly a pharmaceutically or nutraceutically active substance. Yet more particularly it is a macromolecular substance such as a protein or peptide (including enzymes, hormones, antibodies and antigens) or nucleic acid. Other potential active substances include nucleotides, oligo- and poly-nucleotides, nucleosides, vitamins, amino acids, lipids including phospholipids and aminolipids, carbohydrates such as polysaccharides, cells, viruses, steroids, electrolytes, as well as small molecule (ie, non-macromolecule based) actives—in particular water insoluble small molecules—such as pharmaceuticals and imaging agents.

In particular an active substance Z may be hydrophobic, and/or have a low aqueous solubility. As used herein, the terms “water insoluble” and “low aqueous solubility” refer typically to a water solubility of less than 1.0 mg/ml, for instance from 0.1 to 1.0 mg/ml, measured at a physiologically neutral pH for instance from about pH 5.0 to 8.0, and at ambient temperature and pressure. In such cases, conjugation with a hydrophilic PAG moiety such as a PEG group can significantly improve bioavailability on subsequent administration to a human or animal patient.

Suitable biologically active substances Z may be selected from, for example, hypnotics and sedatives, psychic energizers, tranquilizers, respiratory drugs, anti-convulsants, muscle relaxants, anti-parkinson agents (dopamine antagonists), analgesics, anti-inflammatories, anti-anxiety drugs (anxiolytics), appetite suppressants, anti-migraine agents, muscle contractants, anti-infectives (antibiotics, anti-virals, anti-fungals, vaccines) anti-arthritics, anti-malarials, anti-emetics, anepileptics, bronchodilators, cytokines, growth factors, anti-cancer agents, anti-thrombotic agents, anti-hypertensives, cardiovascular drugs, anti-arrhythmics, anti-oxidants, anti-asthma agents, hormonal agents including contraceptives, sympathomimetics, diuretics, lipid regulating agents, anti-androgenic agents, anti-parasitics, anti-coagulants, neoplastics, anti-neoplastics, hypoglycaemics, nutritional agents and supplements, growth supplements, anti-enteritis agents, vaccines, antibodies, diagnostic agents and contrasting agents.

More particularly, the active substance Z may fall into one of a number of structural classes, including but not limited to small molecules (preferably insoluble small molecules), peptides, polypeptides, proteins, polysaccharides, steroids, nucleotides, oligonucleotides, polynucleotides, fats, electrolytes and the like.

Specific examples of active substances suitable for covalent attachment to an activated PAG include but are not limited to aspariginase, amdoxovir (DAPD), antide, becaplermin, calcitonins, cyanovirin, denileukin diftitox, erythropoietin (EPO), EPO agonists (eg, peptides from about 10-40 amino acids in length and comprising a particular core sequence as described in WO-96/40749), dornase alpha, erythropoiesis stimulating protein (NESP), coagulation factors such as Factor VIIa, Factor VIII, Factor IX, von Willebrand factor; ceredase, cerezyme, alpha-glucosidase, collagen, cyclosporin, alpha defensins, beta defensins, exedin-4, granulocyte colony stimulating factor (GCSF), thrombopoietin (TPO), colony stimulating growth factors, alpha-1 proteinase inhibitor, elcatonin, granulocyte macrophage colony stimulating factor (GMCSF), megakaryocyte derived growth factor (MGDF), fibrinogen, filgrastim, growth hormones human growth hormone (hGH), growth hormone releasing hormone (GHRH), GRO-beta, GRO-beta antibody, bone morphogenic proteins such as bone morphogenic protein-2, bone morphogenic protein-6, OP-1; acidic fibroblast growth factors, basic fibroblast growth factor, CD-40 ligand, brain derived neurotrophic factor (BDNF), neurotrophic factor 3 (NT3), neurotrophic growth factor (NGF), heparin, superoxide dismutase, human serum albumin, low molecular weight heparin (LMWH), interferons such as interferon alpha, interferon beta, interferon gamma, interferon omega, interferon tau, consensus interferon; interleukins and interleukin receptors such as interleukin-1 receptor, interleukin-2, interluekin-2 fusion proteins, interleukin-1 receptor antagonist, interleukin-3, interleukin-4, interleukin-4 receptor, interleukin-6, interleukin-8, interleukin-12, interleukin-13 receptor, interleukin-17 receptor; lactoferrin and lactoferrin fragments, luteinizing hormone (LH), luteinizing hormone releasing hormone (LHRH), insulin, pro-insulin, insulin analogues (eg, mono-acylated insulin as described in U.S. Pat. No. 5,922,675), gastrin, prolactin, amylin, C-peptide, somatostatin, somatostatin analogs including octreotide, vasopressin, follicle stimulating hormone (FSH), influenza vaccine, insulin-like growth factor (IGF), insulintropin, macrophage colony stimulating factor (M-CSF), human chorionic gonadotropin (HCG), motilin, plasminogen activators such as alteplase, urokinase, reteplase, streptokinase, pamiteplase, lanoteplase, and teneteplase; nerve growth factor (NGF), kallikrein, osteoprotegerin, adrenocorticotropic hormone (ACTH), platelet-derived growth factor, tissue growth factors, tissue plasminogen activator, transforming growth factor-1, vascular endothelial growth factor, leukemia inhibiting factor, keratinocyte growth factor (KGF), glial growth factor (GGF), glial derived neurotrophic factor (GDNF), T Cell receptors, CD molecules/antigens, tumor necrosis factor (TNF), tumor necrosis factor binding protein (TNF-bp), monocyte chemoattractant protein-1, endothelial growth factors, parathyroid hormone (PTH), thyroid stimulating hormone (TSH), glucagon-like peptide, somatotropin, thymosin alpha 1, thymosin alpha 1 IIb/IIIa inhibitor, thymosin beta 10, thymosin beta 9, thymosin beta 4, alpha-1 antitrypsin, phosphodiesterase (PDE) compounds, VLA-4 (very late antigen-4), VLA-4 inhibitors, bisphosponates, respiratory syncytial virus antibody, cystic fibrosis transmembrane regulator (CFTR) gene, deoxyreibonuclease (DNase), bactericidal/permeability increasing protein (BPI), and anti-CMV antibody. Exemplary monoclonal antibodies include etanercept (a dimeric fusion protein consisting of the extracellular ligand-binding portion of the human 75 kD TNF receptor linked to the Fc portion of IgG1), abciximab, afeliomomab, basiliximab, daclizumab, infliximab, ibritumomab tiuexetan, mitumomab, muromonab-CD3, iodine 131 tositumomab conjugate, olizumab, rituximab and trastuzumab (herceptin).

Additional agents Z suitable for covalent attachment to an activated PAG include but are not limited to amifostine, amiodarone, aminoglutethimide, amsacrine, anagrelide, anastrozole, asparaginase, anthracyclines, bexarotene, bicalutamide, bleomycin, buserelin, busulfan, cabergoline, capecitabine, carboplatin, carmustine, chlorambucin, cisplatin, cladribine, clodronate, cyclophosphamide, cyproterone, cytarabine, camptothecin, 13-cis retinoic acid, all trans retinoic acid; dacarbazine, dactinomycin, daunorubicin, dexamethasone, diclofenac, diethylstilbestrol, docetaxel, doxorubicin, epirubicin, estramustine, etoposide, exemestane, fexofenadine, fludarabine, fludrocortisone, fluorouracil, fluoxymesterone, flutamide, gemcitabine, epinephrine, L-Dopa, hydroxyurea, idarubicin, ifosfamide, imatinib, irinotecan, itraconazole, goserelin, letrozole, leucovorin, levamisole, lomustine, mechlorethamnine, medroxyprogesterone, megestrol, melphalan, mercaptopurine, methotrexate, metoclopramide, mitomycin, mitotane, mitoxantrone, naloxone, nicotine, nilutamide, octreotide, oxaliplatin, pamidronate, pentostatin, pilcamycin, porfimer, prednisone, procarbazine, prochlorperazine, ondansetron, raltitrexed, sirolimus, streptozocin, tacrolimus, tamoxifen, temozolomide, teniposide, testosterone, tetrahydrocannabinol, thalidomide, thioguanine, thiotepa, topotecan, tramadol, retinoin, valrubicin, vinblastine, vincristine, vindesine, vinorelbine, dolasetron, granisetron; formoterol, fluticasone, leuprolide, midazolam, alprazolam, amphotericin B, podophylotoxins, nucleoside antivirals, aroyl hydrazones, sumatriptan; macrolides such as erythromycin, oleandomycin, troleandomycin, roxithromycin, clarithromycin, davercin, azithromycin, flurithromycin, dirithromycin, josamycin, spiromycin, midecamycin, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, grepafloxacin, gatifloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin; aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate; polymixins such as polymixin B, capreomycin, bacitracin, penems; penicillins including penicllinase-sensitive agents like penicillin G, penicillin V; penicillinase-resistant agents like methicillin, oxacillin, cloxacillin, dicloxacillin, floxacillin, nafcillin; gram negative micro-organism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefmetazole, ceftazidime, loracarbef, and moxalactam, monobactams like aztreonam; and carbapenems such as imipenem, meropenem, pentamidine isethiouate, albuterol sulfate, lidocaine, metaproterenol sulfate, beclomethasone dipropionate, triamcinolone acetamide, budesonide acetonide, fluticasone, ipratropium bromide, flunisolide, cromolyn sodium and ergotamine tartrate; taxanes such as paclitaxel; SN-38, and tyrphostines.

The above exemplary active substances are meant to encompass, where applicable, analogues, agonists, antagonists, inhibitors, isomers and pharmaceutically acceptable salt forms thereof. With reference to peptides and proteins, the invention is intended to encompass synthetic, recombinant, native, glycosylated and non-glycosylated forms, as well as biologically active fragments thereof.

The conjugation of such actives with an activated PAG may be used to alter their aqueous solubilities and/or to achieve other effects such as improved stability, reduced toxicity or controlled release on administration. Such PAG conjugates may in particular be suitable for delivery in the form of a solution or suspension, preferably an aqueous solution, such as by injection, orally or by any other suitable route.

The term “PAG conjugate” also encompasses a non-covalent conjugate between an activated PAG and an active substance Z, ie, typically of the general formula (IIIa):

R¹—(O(CH₂)ₙ)ₘ—Y′—Z (IIIa),

where Z is an active substance as described above and Y represents a non-covalent link formed between an activating group X (optionally with a linking group Lᵢ) as defined above and a reactive group in the active substance.

An “active substance” may comprise a substrate on which a PAG derivative is to be immobilised. It may be in any appropriate physical form, for instance microparticles, liposomes or micelles.

Thus an activated PAG may be conjugated, either covalently or non-covalently, to a number of entities including films, chemical separation and purification surfaces, solid supports, metal/metal oxide surfaces such as gold, titanium, tantalum, niobium, aluminium, steel and oxides of such metals, silicon oxides, macromolecules and small molecules. Activated PAGs and their conjugates may be employed for example in biochemical sensors, bioelectronic switches and gates.

An activated PAG may also be conjugated with other polymers, for example thio-reactive polymers, to form for instance polymer matrices or gels. Such matrix or gel products may or may not be cross-linked.

Where the group X is multi-functional, or the activated PAG derivative carries more than one group X, the PAG conjugate may contain a corresponding number of active substance moieties.

Preferably the active substance Z in the conjugate is a protein containing for example:

a) a thiol group —SH (which may itself be formed by reduction of a disulphide bond S—S), such as in a cystine moiety—this may be conjugated to for instance a sulphone group;

b) an amino group —NH₂, such as in a lysine moiety—this may be conjugated to for instance a sulphone, carboxylic acid, active ester (eg, carboxylate, carbonate, sulphonate or isocyanate), aldehyde or aldehyde hydrate, epoxide, mesylate, tosylate, tresylate or carbamide precursor group;

c) an imino group —NH, such as in a histidine moiety—this may be conjugated to for instance a sulphone group; or

d) an acid group —CO₂H— this may be conjugated to for instance a hydroxyl or amine group.

Thus, an active substance may be conjugated to a PAG by means of, without limitation, a (preferably hydrolytically stable) group such as an amide, urethane, amine, imine, carbamate, thioester, ester, carbonate ester, thioether or urea.

A cysteine or other amino acid residue for coupling to an activated PAG may be naturally occurring (ie, it occurs in the protein in its native form) or may be inserted into the native sequence in place of a naturally-occurring amino acid using standard genetic engineering techniques.

The active substance may be conjugated to more than one PAG group R¹—(O(CH₂)ₙ)ₘ via corresponding Y groups.

A PAG derivative or conjugate may have the general formula (IV):

D—(O(CH₂)ₙ)ₘ—D′ (IV),

where D and D′ are each independently either R¹, X or Y—Z (optionally with linking group(s) Lᵢ) as defined above, provided that D and D′ are not both R¹ and that preferably, both D and D′ are selected from X and Y—Z.

A non-linear PAG derivative or conjugate may naturally incorporate more than two groups D/D′.

In particular where the PAG or PAG derivative is a PEG or PEG derivative, or a PPG or PPG derivative, its preferred molecular weight is from 2 to 60 kDaltons, more preferably from 2.5 to 40 kDaltons, still more preferably from 5 to 30 or from 10 to 30 kDaltons, suitably at least 5 kDaltons. Typically, the lower the molecular weight, the lower the preferred operating temperature in the method of the invention, since PAG solubility (for instance in the anti-solvent fluid) tends to increase with decreasing molecular weight. Thus, for instance, for a PEG having a molecular weight of at least 5, preferably at least 10, kDaltons, an operating temperature of 25° C. or lower may be used; for a PEG with a molecular weight below 5 kDaltons, it may be preferable to work at lower temperatures such as from 0 to 10° C., more preferably from 0 to 5° C.

The method of the invention is particularly suitable when the PAG, PAG derivative or PAG conjugate melts or otherwise degrades (by which is meant undergoes any undesired change in physicochemical, in particular physical, form and/or properties) at temperatures as low as 30 or even 25° C. (The term “melts” includes glass formation.) The PAG or derivative or conjugate may for example have a low glass transition temperature T_{g} under the operating conditions used (taking into account if necessary the other reagents present, since CO₂ for instance can lower the T_{g} of, and/or “swell”, polymers such as PEG). A low polymer T_{g} might typically be 30° C. or below, 25° C. or below or 20° C. or below. Polymers suitable for processing using the present invention might have melting points of 70° C. or lower, possibly 65° C. or 60° C. or 55° C. or 50° C. or 45° C. or 40° C. or 35° C. or 30° C. or lower.

In the method of the invention, the target substance preferably comprises no less than 10% w/w of a PAG, PAG derivative and/or PAG conjugate, preferably at least 20% w/w, more preferably at least 30% w/w or 40% w/w or 50% w/w or 60% w/w or 70% w/w. Yet more preferably, the target substance consists essentially of one or more PAGs or derivatives or conjugates thereof (including mixtures of these), by which is meant that the target substance is either a PAG or PAG derivative/conjugate (or a mixture of such materials) with no other substances present, or contains greater than 80% w/w, preferably greater than 90% w/w, more preferably greater than 95% w/w or 98% w/w, of PAGs/PAG derivatives/PAG conjugates.

The invention can provide particular advantages in the production of activated PAGs and PAG conjugates, since it provides a one-step process by which the materials may either be precipitated from solution (for instance, from the solution in which they were synthesised) or even directly synthesised in a dry particulate form. In most cases the need for a subsequent drying step and/or for additional purification steps will be eliminated. There are clear advantages to the use of a one-step process, notably the potential for higher overall yields, greater processing efficiency and lower risks of contamination. Other advantages stemming from the use of a GAS-, in particular a Nektar™ SCF-, type process can include the ability to achieve high purity (in particular with respect to residual solvent levels), dry products with good handling properties and a high degree of control over the physicochemical aspects of the particles formed, for instance their size, size distribution and morphology. The GAS/Nektar™ SCF process also avoids the need for high levels of organic solvents, which can present waste handling and environmental problems, require high processing capacities and contaminate the end product.

Generally speaking, in the method of the invention the temperature at the point where the target solution/suspension contacts the anti-solvent is preferably 20° C. or less, more preferably 10° C. or less, most preferably 5° C. or less, such as about 0° C. or from 4 to −4° C. or from 2 to −2° C. It is preferably no less than −5° C., more preferably no less than 0° C.

To ensure that the anti-solvent fluid is at the desired temperature (or within a desired temperature range) when it contacts the target solution/suspension, it is typically necessary both for the anti-solvent to be at that temperature or within that range immediately before it contacts the target solution/suspension, and also for the temperature to be at the desired level or within the desired range in the region (typically within a particle formation vessel) in which the fluids contact one another and particle formation takes place.

In the method of the invention, the anti-solvent fluid is preferably used in liquid form, and is more preferably above its critical pressure P_{c} under the chosen operating conditions.

Suitable anti-solvents include CO₂, nitrogen, nitrous oxide, sulphur hexafluoride, xenon, ethylene, dimethyl ether, chlorotrifluoromethane, ethane, propane, trifluoromethane and noble gases such as helium, neon or argon. The anti-solvent may comprise a mixture of two or more fluids, so long as the overall mixture is at the appropriate temperature and pressure.

The anti-solvent is preferably CO₂, more preferably liquid CO₂ such as at a pressure of from 75 to 125 bar.

The anti-solvent fluid may optionally contain one or more modifiers (cosolvents), for example water, methanol, ethanol, isopropanol or acetone, which change its ability to dissolve other materials. When used, a modifier preferably constitutes not more than 40 mole %, more preferably not more than 20 mole %, and most preferably from 1 to 10 mole %, of the anti-solvent fluid.

The anti-solvent (together with any modifiers it contains) must be miscible or substantially miscible with the fluid vehicle at the point of their contact, to allow extraction of the vehicle from the target solution/suspension. By “miscible” is meant that the two fluids are miscible in all proportions, and “substantially miscible” encompasses the situation where the fluids can mix sufficiently well, under the operating conditions used, as to achieve the same or a similar effect, ie, dissolution of the fluids in one another and precipitation of the target substance. However the anti-solvent must not to any significant extent, at the point of particle formation, extract or dissolve the target substance. In other words, it should be chosen so that the target substance is for all practical purposes (in particular, under the chosen operating conditions and taking into account any modifiers present) insoluble or substantially insoluble in it. Preferably the target substance is less than 10⁻³ mole %, more preferably less than 10⁻⁵ mole %, soluble in the anti-solvent fluid.

The vehicle is a fluid which is able to carry the target substance in solution or suspension, the term “suspension” including colloidal systems such as emulsions. It may comprise a mixture of two or more component fluids. It may contain, in solution or suspension, other materials apart from the target substance, examples including surfactants, stabilisers, buffers and solubilisers.

The vehicle is preferably a liquid. It is preferably a solvent for the target substance, so that the target substance is carried in solution rather than suspension. The target substance preferably has a solubility (at ambient temperature) of 10⁻³ mole % or greater in the vehicle, more preferably 10⁻² mole % or greater—typical PAGs, in particular PEGs, for use in the invention may for example have an aqueous solubility of 30 to 40% w/v or greater.

Suitable vehicles for PAGs, PAG derivatives and PAG conjugates are organic solvents such as ketones (eg, acetone), halohydrocarbons (eg, dichloromethane (DCM) or chloroform), acetonitrile, tetrahydrofuran (THF) and mixtures thereof. It is preferred that the vehicle be incapable of hydrogen bonding to any significant extent with the ether groups of the PAG; thus for instance it may be preferred for the vehicle not to comprise fluids such as alcohols, dimethyl formamide (DMF) and dimethyl sulphoxide (DMSO).

Water and aqueous solvents may also be suitable, particularly for instance for conjugates of PAGs with water soluble active substances such as proteins. However since water is immiscible with the preferred anti-solvent liquid CO₂, an aqueous vehicle generally necessitates the use of an alternative, water-miscible anti-solvent and/or of an anti-solvent modifier and/or of an additional fluid vehicle as described below.

Moreover for an aqueous vehicle, operating temperatures close to 0° C. may cause the formation of ice crystals and it may therefore be preferred to operate at higher temperatures, for instance at 5° C. or above. Alternatively, again the use of an additional organic solvent vehicle, for instance acetone, may in cases mitigate this problem, especially when the organic vehicle is used in excess.

The concentration of the target substance in the vehicle may suitably be from 0.5 to 40% w/v, preferably from 1 to 25% w/v, depending on the natures of the target substance and vehicle. The concentration should be chosen to ensure an appropriate viscosity in the target solution/suspension, to facilitate its contact with the anti-solvent fluid during the GAS process.

Often it is desirable to use the minimum possible amount of the vehicle to solvate or suspend the target substance, preferably so as to create a single phase solution.

The target solution/suspension may effectively comprise two or more fluids, which may be pre-mixed but which are preferably mixed in situ either substantially simultaneously with or immediately before their contact with the anti-solvent. Such systems are described, eg, in WO-96/00610 and WO-01/03821. The first fluid vehicle may carry the target substance and may for example be water. The second fluid vehicle (which may for example be an organic solvent such as an alcohol or ketone) may be introduced in the anti-solvent flow or separately to the other fluids. It is preferably also introduced in such a manner that it can be dispersed and extracted by the anti-solvent fluid at the same time as the first vehicle, and ideally also in a manner such that the kinetic energy of the anti-solvent can aid in mixing the first and second vehicles. Such fluid contact may be achieved for example using a multi-component coaxial nozzle of the type described in WO-96/00610.

The second vehicle may be a nonsolvent for the target substance, by which is meant that the target substance would typically have a solubility in the second vehicle of less than 10⁻³ mole %, preferably less than 10⁻⁵ mole %. This can help to induce precipitation of the target substance when the second vehicle contacts the target solution/suspension. For instance, the first vehicle may be acetone, tetrahydrofuran or dichloromethane and the second (nonsolvent) vehicle cyclohexane. To further aid precipitation, the second vehicle may contain a “seed” of the target substance, or indeed of any other suitable material (insoluble in the second vehicle), to help induce nucleation of the target substance when the second vehicle comes into contact with the target solution/suspension.

When carrying out this version of the invention, the second vehicle and the target solution/suspension preferably contact one another immediately before their contact with the anti-solvent fluid. The target solution/suspension should also, generally, be highly saturated.

Where two or more fluid vehicles are used, they are preferably miscible or substantially miscible with one another and at least one of them should be miscible with the anti-solvent fluid as described above. The anti-solvent-miscible vehicle(s) are suitably present in excess (relative to the amount of other vehicles present) at the point of anti-solvent contact, so that the anti-solvent can extract all the vehicles together. This may apply for instance if one or more of the vehicles is less than substantially soluble in the anti-solvent fluid (for instance, it has a solubility of 2 or even 1 mole % or less in the anti-solvent).

An excess of one vehicle over another may be achieved by appropriate selection of vehicle flow rates. It can also be desirable for instance when two vehicles are less than fully miscible with one another. Where for example two vehicles are used, the molar ratio of the first to the second may be less than 1:1.5 (for instance, from 1:100 to 1:1.5), preferably less than 1:4, more preferably less than 1:6, most preferably less than 1:9 or 1:10 or 1:20.

Generally speaking however the molar ratio of two vehicles at the point of particle formation may range from 1:99 to 99:1. The relative amounts of all vehicles at this point must of course be chosen so that they are extractable, together, into the anti-solvent fluid. Ideally the amounts are chosen so that, under the operating conditions used, the vehicles form a single phase mixture at the point of particle formation.

Where two or more fluid vehicles are used, they may carry two or more target substances, to be combined in some way at or before the point of particle formation (for instance, to be co-precipitated as a solid dispersion or matrix, or as a simple physical mixture, or one precipitated as a coating around the other, or precipitated as the product of an in situ reaction between the substances). Target substance(s) may also be carried in the anti-solvent fluid.

When carrying out the method of the invention in a particle formation vessel, the temperature and pressure inside the vessel are ideally controlled so as to allow particle formation to occur at or substantially at the point where the target solution/suspension meets the anti-solvent fluid. The conditions in the vessel must generally be such that the anti-solvent fluid, and the solution which is formed when it extracts the vehicle, both remain in a compressed state whilst in the vessel. At the time of particle formation, there should be a single phase mixture of the vehicle(s) and the anti-solvent fluid, to prevent the particulate product being distributed between two or more fluid phases, in some of which it might be able to redissolve.

Selection of appropriate operating conditions will be influenced by the natures of the fluids involved (in particular their solubility and miscibility characteristics) and also by the characteristics desired of the particulate end product, for instance yield, particle size and size distribution, purity and morphology. Variables include the flow rates of the anti-solvent fluid and the target solution/suspension, the concentration of the target substance in the vehicle, the temperature and pressure inside the particle formation vessel, the anti-solvent temperature upstream of the vessel and the geometry of the fluid inlet(s) into the vessel. The method of the invention preferably involves controlling one or more of these variables so as to influence the physicochemical characteristics of the particles formed.

The flow rate of the anti-solvent fluid relative to that of the target solution/suspension, and its pressure and temperature, should be sufficient to allow it to accommodate the vehicle at the point of fluid contact (if applicable, in the presence of appropriate modifier(s)). The anti-solvent flow rate will generally be higher than that of the target solution/suspension—typically, the ratio of the target solution/suspension flow rate to the anti-solvent flow rate (both measured at or immediately prior to the two fluids coming into contact with one another) will be 0.001 or greater, such as 0.003 or 0.005 or greater, preferably from 0.01 to 0.2, more preferably from 0.03 to 0.1. Ideally this ratio will be 0.4 or less, more preferably 0.3 or 0.2 or 0.1 or less.

Thus, the anti-solvent flow rate will generally be chosen to ensure an excess of the anti-solvent over the vehicle when the fluids come into contact, to minimise the risk of the vehicle re-dissolving and/or agglomerating the particles formed. At the point of its extraction, the vehicle may constitute from 1 to 80 mole %, preferably 50 mole % or less or 30 mole % or less, more preferably from 1 to 20 mole % and most preferably from 1 to 5 mole %, of the resultant fluid mixture.

Both the anti-solvent and the target solution/suspension are ideally introduced into the particle formation vessel with a smooth, continuous and preferably pulse-less or substantially pulse-less flow. Conventional apparatus may be used to ensure such fluid flows.

The method of the invention preferably additionally involves collecting the particles following their formation, more preferably in a particle formation vessel into which the fluids are introduced.

According to a second aspect of the present invention, there is provided a method for forming particles of a target substance which consists essentially of one or more polyalkylene glycols (PAGs) or derivatives or conjugates thereof (including mixtures of these), which method comprises carrying out a GAS process, preferably a Nektar™ SCF process, on a solution or suspension of the target substance in a fluid vehicle (the “target solution/suspension”).

The target substance is preferably a single material selected from either a PAG or a PAG derivative or a PAG conjugate.

To our knowledge, although GAS has been used to co-precipitate PAGs, in particular PEGs, with other materials (for instance, as excipients for pharmaceutically active substances), it has not previously been used to precipitate PAGs alone, presumably because it was believed inappropriate for such temperature sensitive materials.

In this context, “consists essentially of” means that the target substance is either a PAG or PAG derivative/conjugate (or a mixture of such materials) with no other substances present, or contains greater than 80% w/w, preferably greater than 90% w/w, more preferably greater than 95% w/w or 98% w/w, of PAGs/PAG derivatives/PAG conjugates.

Preferred features of this second aspect of the invention, for instance with respect to reagents and operating conditions, may be as described above in connection with the first aspect of the invention. In particular, it preferably involves carrying out a GAS-type or Nektar™ SCF-type process but using as the anti-solvent a fluid, preferably a compressed fluid, which at the point of its contact with the target solution/suspension is at a temperature of 25° C. (298 K) or below. The method is preferably applied to a PAG or to an “activated” PAG of the type described above (or to a mixture of PAG and activated PAG). It may in particular be applied to a PEG, a PEG derivative or conjugate or a mixture of such materials.

By “a GAS-type process” in this regard is meant a process which involves contacting the target solution/suspension with a compressed fluid anti-solvent under conditions which allow the anti-solvent to extract the vehicle from the target solution/suspension and to cause particles of the target substance to precipitate from it, as described above, but using as the anti-solvent a fluid which is at a temperature of 25° C. or below, and therefore not necessarily supercritical or near-critical, at the point of its contact with the target solution/suspension.

By “a Nektar™ SCF-type process” (which might also be referred to as “a SEDS™-type process”) is meant a process which involves the introduction of the target solution/suspension and the anti-solvent fluid into a particle formation vessel, under conditions which allow the anti-solvent to disperse the target solution/suspension, and simultaneously to extract the fluid vehicle from it so as to cause particle precipitation, but using as the anti-solvent a fluid which is at a temperature of 25° C. or below, and therefore not necessarily supercritical or near-critical, at the point of its contact with the target solution/suspension. Suitable forms of fluid introduction are described for example in WO-95/01221, WO-96/00610, WO-98/36825, WO-99/44733, WO-99/59710, WO-00/67892, WO-01/03821, WO-01/5664, WO-02/38127 and WO-03/008082, and in co-pending UK patent applications nos. 0300338.1 and 0300339.9, although generally in connection with supercritical or near-critical fluid anti-solvents.

When carrying out the methods of the invention, the target substance may in general be, or be suitable or intended for use in or as, a pharmaceutical or nutraceutical or its excipient; a dyestuff; a cosmetic; a foodstuff; a coating; an agrochemical; a product of use in the ceramics, explosives or photographic industry; etc . . . . It is preferably soluble or substantially soluble in the fluid vehicle, preferably having a solubility in it of 10⁻⁴ mole % or greater under the conditions under which the target solution is prepared (ie, upstream of the point of particle formation).

In particularly preferred embodiments of the invention, the target substance is for use in or as a pharmaceutical or pharmaceutical excipient, or in or as a nutraceutical or nutraceutical excipient.

The target substance may be in a single or multi-component form (eg, it could comprise an intimate mixture of two materials, or one material in a matrix of another, or one material coated onto a substrate of another, or other similar mixtures). It may comprise two associated substances, for example in a covalently bonded conjugate such as the PAG-active substance complexes described above.

The methods of the invention may be used selectively to precipitate one or more target substances from a mixture of substances. The operating temperature and pressure, and the vehicle and anti-solvent, may be chosen so that only the target substance(s) precipitate whereas the other components of the mixture (which may, for example, be impurities) are extracted into the anti-solvent with the vehicle; this depends naturally on the solubility of the target substance(s) and other component(s) in the vehicle and anti-solvent under the chosen operating conditions.

Thus, the present invention may be used for example to purify a substance from a mixture containing that and other substances.

In particular, it may be used selectively to precipitate a target “activated” PAG from a reaction mixture containing the target as well as impurities such as unreacted PAG base polymer. It may be used selectively to precipitate a PAG-active substance conjugate from a reaction mixture which also contains, for instance, unconjugated starting materials.

The methods of the invention may be used to coformulate two or more target substances to produce a multi-component product. Such a product may for example comprise an intimate mixture of two or more materials, such as in a solid dispersion, or a physical mixture of two or more materials, or one material coated onto a substrate of another, or a mixture of such physical forms. Examples include two pharmaceutically active moieties intended for co-administration, or a pharmaceutical together with an excipient.

The two or more target substances may be co-introduced in a common fluid vehicle, or in separate fluid vehicle(s) which contact one another at or immediately before the point of anti-solvent contact. Target substance(s) may also be carried in the anti-solvent itself.

In particular, the methods of the invention may be used to coformulate an active substance, such as a pharmaceutically or nutraceutically active substance, with a PAG or PAG derivative.

Within the coformulated product, the target substances may be associated with one another for instance via some form of chemical or physical interaction. In particular they may be associated in the form of a complex or conjugate or other reaction product. Alternatively, they may be present as distinct entities.

According to a third aspect of the invention, there is provided a particulate product formed using a method according to the first or the second aspect. This may in particular consist essentially of a PAG or PAG derivative or PAG conjugate.

A PAG or PAG derivative/conjugate formed using a method according to the present invention, in particular a PEG or PEG derivative/conjugate, can often have good physicochemical particle properties. It may for instance be produced in the form of a dry powder, usually easily handlable and free flowing and with good shelf storage stability. Small particles, for instance of volume mean diameter 30 μm or less, preferably 26 or 25 μm or less, more preferably 20 μm or less or even 15 μm or 10 μm or 7 μm or 5 μm or less, may be produced, and with a relatively narrow size distribution (for instance with a particle size spread of 2.4 or less, such as from 1.5 to 2.4, preferably 2 or less, more preferably 1.8 or 1.7 or less). (Particle size “spread” is defined as (D₉₀−D₁₀)/D₅₀ where D is the diameter of the relevant particle population.) In cases, volume mean particle diameters may range from 1 to 15 μm, preferably from 2 to 12 μm, more preferably from 4 to 10 μm. The particles of the invention are typically only loosely, if at all, agglomerated, in contrast to PAGs and their derivatives and conjugates made by more conventional non-GAS techniques which tend to be highly agglomerated and often “sticky” in nature.

Typically the methods of the invention may be used to reduce the particle size of the target substance to 90 or 80 or 70% or less of that of the starting material, preferably to 50% or less of that of the starting material, more preferably to 40% or 30% or 20% or even 10% or less.

Particle sizes may be measured for instance using (a) an Aerosizer™ time-of-flight instrument (which gives an aerodynamic equivalent particle diameter (mass median aerodynamic diameter, MMAD)) or (b) a laser diffraction sensor such as the Helos™ system available from Sympatec GmbH, Germany (which provides a geometric projection equivalent (mass median diameter, MMD)). MMADs may also be assessed using a cascade impactor. Volume mean diameters may be obtained in both cases using commercially available software packages.

Particles produced according to the invention also tend to possess better dissolution properties (ie, a greater speed and/or efficiency of dissolution) in aqueous solutions than corresponding products made by prior art techniques. In particular PEGs and PEG derivatives/conjugates produced by non-GAS techniques such as freeze drying often suffer from poor dissolution characteristics, which can present problems if (as is likely where a PEG is to be used as an excipient for instance for an injectable pharmaceutical) they need to be formulated in high concentrations.

In the case where the product of the invention is a conjugate of a PAG or PAG derivative with an active substance, in particular a PEG conjugate, it preferably has an aqueous solubility of at least 300 mg/ml, more preferably at least 400 mg/ml, at room temperature (typically from 18 to 25° C., such as 22 or 25° C.).

The product of the present invention will typically contain less than 2000 ppm of residual solvent. It preferably contains less than 1000 or 500 ppm, more preferably less than 200 ppm, most preferably less than 150 or 100 or even 50 ppm residual solvent, by which is meant solvent(s) which were present at the point of particle formation, for instance in the target solution/suspension and/or the anti-solvent fluid. Still more preferably the product contains no detectable residual solvent, or at least only levels below the relevant quantification limit(s).

Such low residual solvent levels may be achieved, according to the invention, simply by forming the product using a single step GAS or more preferably a Nektar™ SCF particle formation process, without the need to subject the direct product of that process to a subsequent period of drying. By “drying” in this context is meant generally evaporative drying, in air, usually at a temperature higher than ambient (say, higher than 22 or 25° C.) and typically in an oven, such as at a temperature of 30 or 35 or even 40° C. or higher, for a period of for example an hour or more, typically 6 or 12 or 24 or in some cases 36 or 48 hours or more—such drying steps are often needed when PAGs and related products are formed by more conventional, non-GAS, processes involving precipitation or crystallisation from an organic solvent and subsequent filtration.

Since the products of the invention can be produced without oven drying, in general smaller and more uniform particle sizes are possible. During higher temperature drying, there can be a tendency for particles to melt and coalesce, for instance if heating is less than completely uniform and local “hot-spots” arise within the product. This is a particular problem for relatively low melting point polymers such as PAGs and in particular PEGs, and can result in coarser and often less free flowing particles. Moreover, the evaporation of solvent during drying, from within the body of a particle, can lead to cavities in the particle especially at or near its surfaces; the effect on surface morphology (a less smooth, typically higher energy, surface) can in turn increase the risk of inter-particle adhesion and compromise flowability, dispersibility in fluids and other handling characteristics. Also higher temperature drying may compromise the integrity of any temperature sensitive active substances included in a particulate product, as well as (being an additional processing step) increasing the potential for product contamination and loss of yield.

Particulate products according to the invention, in contrast, tend to have smooth and relatively low energy surfaces, typically less adhesive than those of corresponding products made by prior art techniques. They also tend to be in the form of solid—eg, as opposed to hollow, porous (which includes perforated, or cavity-containing) or at least partially fluid-containing—particles.

Generally the product of the invention preferably contains 2.5% w/w or less, more preferably 2 or 1.5 or 1% w/w or less of impurities, by which is meant substances (either solid or liquid phase) other than the target substance intended to be formed into particles.

Where the product contains an active, in particular a biologically and/or pharmaceutically active, substance and more particularly a proteinaceous material, the active substance preferably retains at least 90%, more preferably at least 95%, of its original activity after processing into particles using a method according to the invention, or regains that activity on resolvation. “Original activity” may here mean the activity of the active substance alone, ie, unconjugated, in the absence of excipients and prior to processing in accordance with the invention, and/or in the case where the target substance is itself a PAG-active substance conjugate, the activity of the active substance within the conjugate prior to processing according to the invention.

The active substance preferably also retains, or substantially retains, its chemical integrity following processing according to the invention, which may include its structural properties in particular where it has a secondary and/or tertiary structure as in the case of proteinaceous active substances.

The present invention provides, according to a fourth aspect, any polyalkylene glycol (PAG) or a derivative or conjugate thereof, in particulate form, having one or more of the above described properties which may be obtained by preparing the product using a GAS or more preferably a Nektar™ SCF particle formation process. In particular, such a PAG or derivative or conjugate preferably has a residual solvent content of 2000 ppm or less, more preferably of 1000 or 500 or 200 or 150 or 100 or 50 ppm or less; and/or a volume mean diameter of 30 μm or less, more preferably 25 or 20 or 15 or 10 μm or less; and/or a volume mean diameter spread of 2.4 or less, preferably 2 or less; and/or an impurity content of 2.5% w/w or less; and/or an aqueous solubility of at least 300 mg/ml at room temperature; and/or is in the form of a free flowing powder, preferably non- or only loosely agglomerated, typically made up of solid particles.

Such products may often be produced, using single-step precipitation methods according to the invention, in yields of 50% w/w or higher, preferably 55 or 58 or 60 or 65 or even 70 or in cases 80% w/w or higher.

The above properties may also apply to a particulate coformulation of (i) a PAG, or a derivative or conjugate thereof, with (ii) an active substance such as a pharmaceutically active substance. Such a coformulation may take the form, for example, of an intimate solid dispersion of components (i) and (ii), or of a coating of one component on the other. It may be prepared according to the invention by using a GAS—preferably a Nektar™ SCF—particle formation process in which a compressed fluid anti-solvent is used simultaneously both to extract a fluid vehicle from, and to disperse, a solution or suspension of both components (i) and (ii) in the vehicle so as to co-precipitate the two components.

A fifth aspect of the invention provides a composition containing one or more products according to the third or the fourth aspect, in particular a PAG-active substance conjugate. Such a composition may in particular be a pharmaceutical or nutraceutical composition, which contains a product according to the invention together with a pharmaceutically and/or nutraceutically acceptable excipient. The composition may be in a solid or liquid form.

Pharmaceutical formulations according to this fifth aspect of the invention will typically contain, in addition to the PAG or PAG derivative or conjugate, one or more pharmaceutically acceptable carriers, excipients, solvents, stabilizers, etc, depending upon the intended mode of adminstration and dosage form. Pharmaceutical excipients and/or additives suitable for use in the compositions of the invention are described in “Remington: The Science & Practice of Pharmacy”, 19^{th} edition, Williams & Williams (1995), in the “Physician's Desk Reference”, 52^{nd} edition, Medical Economics, Montvale, N.J., USA (1998) and in Kibbe, A H, “Handbook of Pharmaceutical Excipients”, 3^{rd} edition, American Pharmaceutical Association, Washington D.C., USA (2000).

Conjugate-containing formulations for parenteral administration are most typically liquid solutions or suspensions, while inhaleable formulations for pulmonary administration are generally liquids or powders, with powder formulations being generally although not necessarily preferred. Additional albeit less preferred conjugate compositions include syrups, creams, ointments, tablets and the like.

PAG-active substance conjugates such as those described above can be administered parenterally by intravenous injection, or less preferably by intramuscular or by subcutaneous injection. Suitable formulation types for parenteral administration include ready-for-injection solutions, dry powders for combination with a solvent prior to use, suspensions ready for injection, dry insoluble compositions for combination with a vehicle prior to use, emulsions and liquid concentrates for dilution prior to administration.

Because embodiments of the present invention are modified versions of the inventions disclosed in WO-95/01221, WO-96/00610, WO-98/36825, WO-99/44733, WO-99/59710, WO-01/03821, WO-01/15664, WO-02/38127 and WO-03/008082, and in co-pending UK patent applications nos. 0300338.1 and 0300339.9, technical features described in those documents, for instance regarding the selection of appropriate reagents, operating conditions and processing apparatus, can apply also to the present invention. The eleven earlier documents are therefore intended to be read together with the present application.

In this specification the term “substantially”, when applied to a condition, is meant to encompass the exact condition (eg, exact simultaneity) as well as conditions which are (for practical purposes, taking into account the degree of precision with which such conditions can be measured and achieved) close to that exact condition, and/or which are similar enough to that exact condition as to achieve, in context, the same or a very similar effect. In particular, “substantially simultaneously” and “substantially immediately”, referring to the timing of fluid contact events, imply sufficiently small time intervals (for instance, between the anti-solvent fluid contacting the vehicle(s), and the fluids entering a particle formation vessel) as preferably to eliminate, or substantially eliminate, the risk of particle formation occurring upstream of the particle formation vessel. Where two vehicles are to be mixed immediately before their contact with the anti-solvent fluid, the timing of their contact with one another, relative to that of their dispersion by the anti-solvent, will depend on the nature of the fluids (in particular the degree of immiscibility of the two vehicles), the target substance and the desired end product, as well as on the size and geometry of the particle formation vessel and the fluid inlet(s) and on the fluid flow rates. The contact may occur within 0 to 1000 milliseconds, preferably within 0.001 to 100 or 0.001 to 10 milliseconds, more preferably within 0.1 to 10 milliseconds, still more preferably within 1 to 7 milliseconds, of the dispersion.

References to solubilities and miscibilities, unless otherwise stated, are to the relevant fluid characteristics under the operating conditions used.

References to “operating conditions” are to the conditions (including temperature, pressure and the natures and amounts of the fluids (including modifiers) and other reagents present) under which particle formation occurs, ie, generally the conditions under which the target solution/suspension contacts the anti-solvent fluid, which is preferably at or substantially at the point where the fluids enter the vessel in which particle formation is to take place.

The present invention will now be illustrated by means of the following examples and with reference to the accompanying illustrative drawings, of which:

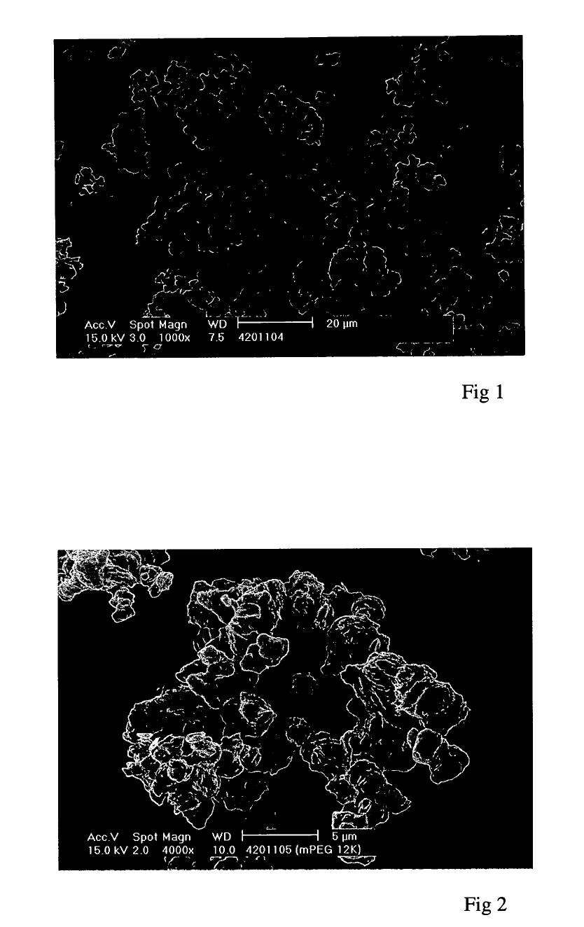

FIGS. 1 to 8 are scanning electron microscope (SEM) photographs of the products of some of Examples 1 to 8 below.

### DETAILED DESCRIPTION

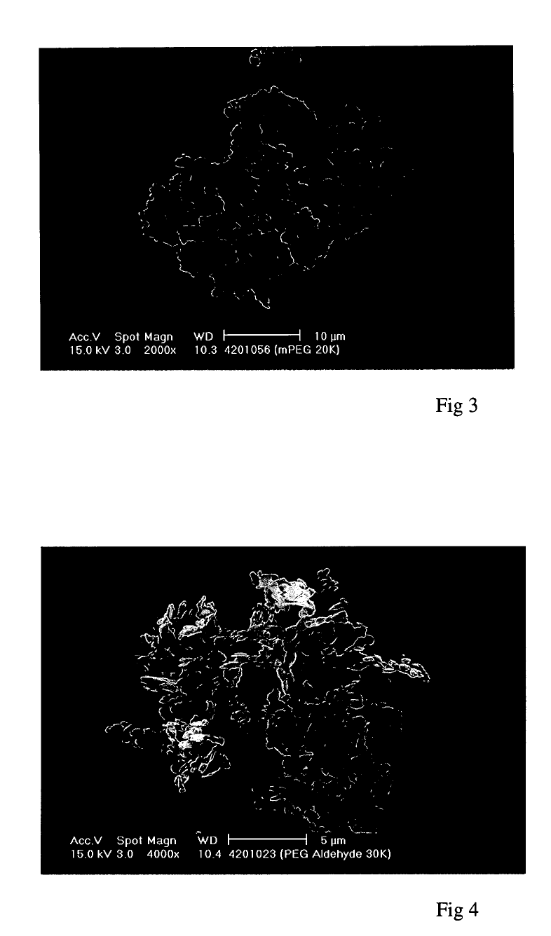

These examples, which relate to the precipitation of PEGs, activated PEGs, PEG conjugates and PEG/PPG copolymers, were carried out using a Nektar™ SCF (SEDS™) particle formation system of the general type shown schematically in FIG. 1 of WO-95/01221. A two- or in some cases three-passage coaxial nozzle (see FIGS. 3 and 4 of WO-95/01221) was used to co-introduce into a particle formation vessel (i) a solution of the target substance in a fluid vehicle, (ii) liquid CO₂ and in some cases (iii) a second fluid vehicle. The nozzle had a convergent tip and its inner passage(s) terminated a short distance upstream, in the direction of fluid flow, of the outlet of its outer passage.

The temperature and pressure were controlled within the particle formation vessel to ensure the CO₂ remained in liquid form throughout the process. Particle formation occurred at the nozzle outlet, where the fluids came into contact immediately before entering the vessel; here the CO₂, introduced with a much higher flow rate than that of the target solution, acted both to disperse and to mix the fluids and also to extract the vehicle(s) thus precipitating the target substance.

The operating pressure within the vessel was 100 bar in all experiments. The operating temperature (ie, that of the CO₂ on entering the particle formation vessel, and also within the vessel itself) was in most cases 25° C.; similar if not better results could be expected at lower temperatures.

The outlet diameter of the nozzle used was, unless otherwise stated, 200 μm. The CO₂ flow rate (measured at the pump head) was generally 20 ml/min, that for the target solution usually 0.1 ml/min.

The solvents used were acetone, tetrahydrofuran (THF), dichloromethane (DCM) and ethyl acetate. In certain cases, cyclohexane was used as a second (anti-solvent) vehicle. Wherever possible, the target solutions were made up in saturated form.

Since many PEGs have a tendency to gel in solution in organic solvents if not kept warm, especially if saturated, the PEG target solutions were maintained where possible at between 25 and 35° C. To mitigate problems caused by high target solution viscosity, in cases a stainless steel loop of volume between 3 and 29 ml was inserted between the pump outlet and the oven inlet for the solution; this loop was pre-loaded manually with the target solution and on commencement of the particle formation run, neat solvent was pumped through the loop thus forcing the target solution into the pressure vessel.

All particle sizes (quoted as volume mean diameters) and spreads were measured using an Aerosizer™ at 2 bar shear pressure, unless otherwise stated.

All PEGs, PEG derivatives and PEG conjugates used were sourced from Shearwater Corporation, Huntsville Ala., US.

Examples 1 demonstrate the formation of particulate PEGs using a method according to the present invention. In Examples 2, 3 and 9, “activated” PEG derivatives are precipitated by the same method. Examples 4 to 6 show the precipitation of PEG-active substance conjugates in accordance with the invention. Examples 7 show the precipitation of a PEG/PPG copolymer, Examples 8 the precipitation of a range of PEG-protein conjugates.

### EXAMPLES 1

Linear PEGs of formula (I) (R¹=methyl), of different molecular weights, were successfully precipitated using a Nektar™ SCF-type process according to the method of the invention.

The 5 kDalton (±500 dalton) PEG starting material had a particle size of 27.3 μm and a melting point of 64° C. and contained ≦1% of the non-methylated base diol. The 12 kDalton (±1200 dalton) starting material had a particle size of 35.1 μm and a melting point of 65.7° C. and contained ≦1.5% of the non-methylated diol. The 20 kDalton (±200 dalton) PEG starting material had a particle size of 46.9 μm, a melting point of 66.6° C. and a diol content of ≦3%.

The CO₂ flow rate was 20 ml/min (measured at the pump head), that for the target solution 0.1 ml/min. A 50 ml particle formation vessel was used.

Different solvents and target solution concentrations were tested, as set out in Table 1 below, together with the product yields and particle sizes. In all cases the product was a fine free-flowing powder with a relatively narrow particle size distribution.

TABLE 1

| | PEG | | PEG | | Product | | Run |
|---|---|---|---|---|---|---|---|
| Experi- | molecular | | solution | | particle | Particle | dura- |
| ment | weight | | concⁿ | Yield | size | size | tion |
| no. | (kDaltons) | Solvent | (% w/v) | (%) | (μm) | spread | (min) |
| 1.1 | 5 | Acetone | 6 | 73 | 4.8 | 1.5 | 36 |
| 1.2 | 5 | THF | 7.5 | 71 | 11.6 | 1.7 | 32 |
| 1.3 | 12 | Acetone | 6 | 59 | 5.9 | 1.6 | 35 |
| 1.4 | 12 | THF | 7.5 | 82 | 6.3 | 1.6 | 110 |
| 1.5 | 20 | Acetone | 15 | 93 | 7.863 | 2.3 | 80 |
| 1.6 | 20 | DCM/ | 6 | 67 | 5.366 | 2 | 70 |
| | | cyclo- | | | | | |
| | | hexane | | | | | |
| | | mixture | | | | | |
| | | (45:55) | | | | | |
| 1.7 | 20 | DCM | 33 | 60 | 17.11 | 1.8 | 30 |

In a further experiment, no. 1.8, the 20 kDalton PEG was precipitated from THF (10% w/v) in the presence of cyclohexane. The target solution and the cyclohexane were co-introduced into the particle formation vessel via separate passages of the three-component coaxial nozzle, so as to contact one another only immediately before their contact with the CO₂. The cyclohexane flow rate was 0.5 ml/min and the run duration 70 minutes; all other operating conditions were the same as for Experiments 1.1 to 1.7. In this case the product yield was 72.2%, and its particle size was 4.665 μm (Sympatec™ particle size 4.7 μm) with a spread of 1.8.

FIGS. 1 to 3 are SEM photographs of the products of Experiments 1.1, 1.4 and 1.8 respectively.

### EXAMPLES 2

These experiments demonstrate the precipitation, using a Nektar™ SCF-type method according to the invention, of an “activated” PEG. The starting material was a linear PEG of formula (II) in which R¹=methyl and X=CHO (at least 80% aldehyde groups, ie, ≦20% unactivated PEG). It had a molecular weight of 30 kDaltons (±3,000 dalton), a particle size of 55.6 μm and a melting point of 59.9° C. It contained residual solvents DCM and isopropyl alcohol (IPA) in total amounts less than 100 ppm, however these levels had been achieved by oven drying. It had the form of agglomerated flakes.

Attempts to precipitate this material from methanol, a methanol/ethanol (1:1) mixture and dichloromethane at supercritical temperatures (80, 60 and 40° C.) did not yield a particulate product, presumably due to the solubility of the PEG in the CO₂ anti-solvent under such conditions. In some cases this resulted in no yield at all, in others the PEG deposited in the form of a waxy as opposed to a fine particulate solid.

The activated PEG was however successfully precipitated from various solvent systems using an operating temperature of 25° C. and an operating pressure of 100 bar, as shown in Table 2 below. The nozzle outlet diameter was 200 μm for all except Experiment 2.6, in which it was 500 μm. The vessel volume was 50 ml for Experiments 2.1 to 2.4, and 500 ml for Experiments 2.5 to 2.8.

Where two vehicles were used, they were introduced via separate passages of a three-component coaxial nozzle, as described in connection with Experiment no. 1.8.

Table 2 also shows the yields and particle sizes for the products, all of which were in the form of fine free flowing powders with narrow particle size distributions. FIG. 4 is a SEM photograph of the product of Experiment 2.1.

TABLE 2

| | | | PEG | PEG | 2^{nd} | | | Product | | |
|---|---|---|---|---|---|---|---|---|---|---|
| | | | solution | solution | vehicle | CO₂ flow | | particle | Particle | Run |
| Expt | 1^{st} | 2^{nd} | concⁿ (% | flow rate | flow rate | rate | Yield | size | size | duration |
| no. | vehicle | vehicle | w/v) | (ml/min) | (ml/min) | (ml/min) | (%) | (μm) | spread | (min) |
| 2.1 | #A | — | 1 | 0.1 | — | 20 | 64 | 3.076 | 1.7 | 110 |
| 2.2 | #B | — | 15 | 0.1 | — | 20 | 62 | 7.128 | 1.9 | 90 |
| 2.3 | #B | #C | 15 | 0.1 | 1 | 20 | 64 | 10.01 | 1.7 | 66 |
| 2.4 | #D | #C | 5 | 0.1 | 0.5 | 30 | 99 | 12.2 | 2 | 300 |
| 2.5 | #D | #C | 5 | 0.2 | 0.5 | 20 | 94 | 8.31 | 2.1 | 150 |
| 2.6 | #A | — | 15 | 0.1 | — | 20 | 73 | 16.1 | 1.6 | 300 |

(Key to vehicles: #A = acetone; #B = DCM; #C = cyclohexane; #D = DCM/acetone mixture (1:49 v/v)).

Experiment no. 2.1 was repeated (run duration 80 minutes) at an operating temperature of 0° C., and again a particulate product was successfully obtained, having a particle size of 33 μm and size spread of 1.7. The yield was 62%.

Residual solvent levels were measured for the products of Examples 2.4 and 2.5. In both cases, no DCM or cyclohexane were detected, and the acetone levels were below the 150 ppm quantification limit.

### EXAMPLES 3

In these experiments, a Nektar™ SCF-type process was used to precipitate a branched two-arm activated PEG of formula (V) below:

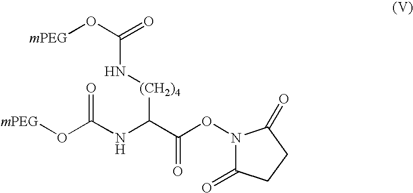

in which mPEG represents a linear PEG chain as defined in formula (I) with R¹ being methyl and —O—R² being replaced by the N-hydroxysuccinimidyl ester/amide linkage group. This is a branched PEG of which the two PEG arms are connected via urethane linkages to a lysine linker activated as the N-hydroxysuccinimide (“NHS”) ester, ie, mPEG2-N-hydroxysuccinimide.

The activated PEG has a total molecular weight of 40 kDaltons (20 kDaltons for each mPEG arm). Its “apparent” molecular weight is expected to reflect that of each arm, ie, in this case 20 kDaltons (±4,000 dalton). The starting material had a particle size of 33.5 μm and a melting point of 60.9° C., and contained the residual solvents DCM and IPA in total amounts less than 100 ppm.

It was precipitated from DCM (15% w/v; solution flow rate 0.1 ml/min). Cyclohexane was co-introduced (flow rate 1 ml/min) with the PEG solution and CO₂ via a three-component nozzle, as described in connection with Experiment 1.8 above.

The operating temperature was 25° C. and the pressure 100 bar. The nozzle outlet diameter was 500 μm and the vessel volume 500 ml. The run duration was 120 minutes.

The experiment resulted in a free flowing powder, with a yield of 63%. The product particle size was 18.81 μm (spread 1.7).

### EXAMPLES 4

A conjugate of mPEG (ie, a linear PEG chain as defined in formula (I) in which R¹=methyl) with dipalmitoyl phosphatidyl ethanolamine (DPPE) was precipitated from ethyl acetate using the method of Examples 1 to 3. The structure of the ethanolamine-mPEG link was:

—CH₂—CH₂—NH—CO₂-mPEG.

The molecular weight of the PEG was 5 kDaltons, that of the conjugate 5750±600 daltons. The particle size of the starting material was 8.9 μm and its melting point 54° C. It had a purity of >97% and contained the residual solvents chloroform, acetonitrile and IPA in total amounts less than 100 ppm.

The PEG solution concentration was 2.5% w/v. Its flow rate into the particle formation vessel was 0.1 ml/min and the CO₂ flow rate was 20 ml/min. The operating temperature was 25° C. and the pressure 100 bar. The nozzle outlet diameter was 200 μm, the vessel volume 50 ml.

In Experiment 4.1, using a run duration of 30 minutes, a fine free flowing powder was obtained with 41% yield. The particle size was 7.6 μm. In Experiment 4.2, using a run duration of 132 minutes, a fine free flowing powder was obtained with 56% yield. Here the particle size was 9.0 μm, spread 1.9. FIG. 5 is a SEM photograph of the Experiment 4.1 product.

Attempts to precipitate the PEG conjugate at 35° C., all other conditions the same, were unsuccessful.

### EXAMPLE 5

A conjugate of mPEG (ie, a linear PEG chain as defined in formula (I) in which R¹=methyl) with distearoyl phosphatidyl ethanolamine (DSPE) was precipitated from DCM using the method of Examples 1 to 3. The structure of the DSPE-mPEG link was as for the conjugate of Examples 4. The molecular weight of the PEG was 2 kDaltons. The particle size of the starting material was 12.3 μm and its melting point 50.6° C. It had a purity of >97% and contained the residual solvents chloroform, acetonitrile and IPA in total amounts less than 100 ppm.

The PEG solution concentration was 2.5% w/v, its flow rate 0.1 ml/min. The CO₂ flow rate was 20 ml/min. The operating temperature was 25° C. and the pressure 100 bar. The nozzle outlet diameter was 200 μm, the vessel volume 50 ml. The run duration was 30 minutes.

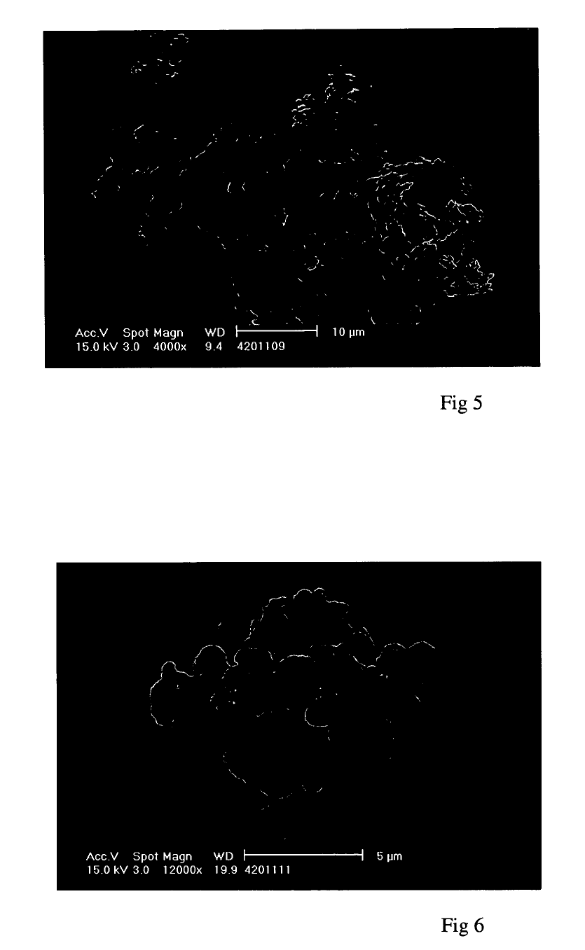

A fine free flowing powder was obtained with 52% yield. The particle size was 7.6 μm, standard deviation 2.1. FIG. 6 is a SEM photograph of the product.

Again attempts to precipitate the conjugate at 35° C. were unsuccessful.

### EXAMPLES 6

An ester-linked conjugate of mPEG (ie, R¹=methyl) with R-trans-retinoic acid (ATRA) was precipitated from acetone using the method of Examples 1 to 3. The molecular weight of the PEG was 5 kDaltons. The particle size of the starting material was 20.0 μm (standard deviation 1.9), its melting point 54.7° C.

The conjugate solution concentration used was 5% w/v, its flow rate 0.1 ml/min. The CO₂ flow rate was 20 ml/min. The operating temperature was 25° C. and the pressure 100 bar. The nozzle outlet diameter was 200 μm, the vessel volume 50 ml. The run duration was 30 minutes.

A fine free flowing powder was obtained with 33% yield. The particle size was 7.38 μm, standard deviation 1.83.

In a further experiment, using a run duration of 120 minutes but with all other operating conditions the same, a particulate product was obtained in a yield of 78%, particle size 5.92 μm, standard deviation 1.55.

Using supercritical processing temperatures (ie, above 31° C.) did not result in successful precipitation of the PEG-ATRA conjugate.

### EXAMPLES 7

A commercially available PEG/PPG copolymer, Pluronic™ F127 (average molecular weight 12.6 kDaltons) was precipitated from a 1:9 (v/v) DCM/acetone mixture. Pluronic™ F127 contains 70% PEG and 30% PPG. The starting material had a melting point of 56° C.

The Pluronic™ solution concentration used was 5% w/v, its flow rate 0.4 ml/min. The operating temperature was 25° C. and the pressure 100 bar. The nozzle outlet diameter was 200 μm, the vessel volume 50 ml.

In Experiment 7.1 the CO₂ flow rate was 20 ml/min; in Experiment 7.2 it was 10 ml/min.

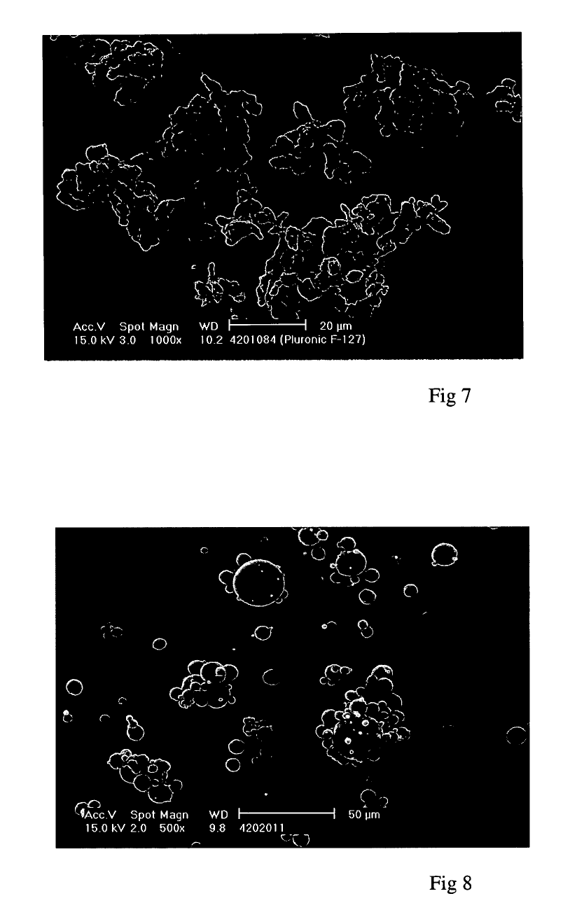

Fine free flowing powders were obtained, with yields of 68% and 66% for Experiments 7.1 and 7.2 respectively. FIG. 7 shows a SEM photograph of the product of Experiment 7.2.

### EXAMPLES 8

Conjugates of the protein lysozyme with linear mPEGs of various molecular weights were processed from aqueous solution using the method of the invention. All conjugates were obtained from Shearwater Corporation, Huntsville Ala., US.

The operating temperature was 0° C. and the pressure 100 bar. Each target solution contained 20 mg/ml conjugate. Acetone was used as a second (non-solvent) vehicle. The flow rates were 0.03 ml/min for the target solutions, 5 ml/min for the acetone and 20 ml/min for the liquid CO₂ anti-solvent.

Unconjugated lysozyme was also subjected to Nektar™ SCF processing, again from a 20 mg/ml aqueous solution in the presence of acetone, using the same operating conditions as for the PEG conjugates.

The results are shown in Table 3.

TABLE 3

| | MPEG | | | Starting material |
|---|---|---|---|---|
| Experiment | molecular weight | Yield | Particle size | particle size |
| no. | (kDaltons) | (%) | (μm) | (μm) |
| 8.1 | N/A | 20 | 14 | 17.8 |
| | (unconjugated | | | |
| | lysozyme) | | | |
| 8.2 | 5 | 67 | 16.5 | 7.9 |
| 8.3 | 20 | 42 | 14.3 | 13.4 |
| 8.4 | 40 | 28 | 16.5 | 18.7 |

SEM observation of the Nektar™ SCF products revealed them to be smooth approximately spherical particles as compared to rough and broken plate-like particles for the conjugate starting materials. FIG. 8 is a SEM photograph of the product of Experiment 8.3, for example.

The Nektar™ SCF-produced lysozyme was found to have retained 105% of its original (ie, pre-Nektar™ SCF) activity, and the 5 kDalton PEG-lysozyme conjugate 108%.

### EXAMPLE 9

A Nektar™ SCF process as in Examples 1 to 3 was used to produce particles of a branched 8-arm activated PEG. Each arm comprised a linear PEG chain of molecular weight 2.5 kDaltons terminating in an —NH₂ group; the total molecular weight of the activated PEG was therefore approximately 20 kDaltons. The particle size of the starting material was too large to be feasibly measured using the Aerosizer™; SEMs however showed rough-surfaced particles with dimensions greater than 50 μm. The melting point was 54° C.

The target PEG was precipitated from THF (solution concentration 5% w/v; solution flow rate 0.4 ml/min). The CO₂ flow rate was 20 ml/min. The operating temperature was 0° C. and the pressure 100 bar. The nozzle outlet diameter was 200 ml, the vessel volume 50 μm. The run duration was 25 minutes.

A fine free flowing powder, particle size 10.3 μm, was obtained with 76% yield.

1. A composition selected from the group consisting of a polyalkylene glycol (PAG), a PAG derivative and a PAG conjugate, wherein said composition is in particulate form, and wherein said composition has a residual solvent content of 200 ppm or less and a volume mean particle diameter of 25 μm or less.

2. The composition of claim 1, wherein the residual solvent content is 150 ppm or less.

3. The composition of claim 2, wherein the residual solvent is not detectable.

4. The composition of claim 1, wherein the volume mean particle diameter is 10 μm or less.

5. The composition of claim 1, wherein the particles have a volume mean diameter spread of 2.4 or less.

6. The composition of claim 1 which is in the form of a free flowing powder.

7. The composition of claim 1, which contains 2.5% w/w or less of impurities.

8. The composition of claim 1, which is a PAG or a PAG derivative.

9. The composition of claim 8, which is an activated PAG containing one or more reactive functional groups X on the main polymer chain(s) and/or at its terminae, where X is an activating group which facilitates subsequent conjugation of the polymer to another substance.

10. The activated PAG of claim 9, wherein X is selected from the group consisting of an ester, aldehyde, aldehyde precursor, acetal, maleimide, benzotriazole carbonate, carboxyl and derivatives thereof.

11. A composition comprising a covalently bound conjugate of an activated PAG, and an active substance Z, said composition in particulate form wherein said composition has a residual solvent content of 200 ppm or less and a volume mean particle diameter of 25 μm or less.

12. The composition of 11, wherein the active substance Z is selected from the group consisting of pharmaceutically and nutraceutically active substances.

13. The composition of claim 12, wherein the active substance Z is selected from the group consisting of proteins, peptides and polypeptides.

14. The composition of claim 11, wherein the active substance Z has a low aqueous solubility.

15. The composition of claim 11, which has an aqueous solubility of at least 300 mg/ml at room temperature.

16. The composition of claim 12, wherein the active substance Z is a pharmaceutically active substance and retains at least 95% of its original activity after having been processed into particles, or regains that activity on resolvation of the particulate conjugate.

17. The composition of claim 1 selected from the group consisting of polyethylene glycol (PEG) and derivatives and conjugates thereof.

18. The composition of claim 1, which has been prepared in particulate form using a GAS particle formation process.

19. The composition of claim 18, which has been prepared in particulate form using a Nektar™ SCF particle formation process in which a compressed fluid anti-solvent is used simultaneously both to extract a fluid vehicle from, and to disperse, a solution or suspension containing composition in the vehicle.

20. The composition of claim 18, which has been prepared in particulate form using a GAS particle formation process and which has not been subjected to subsequent evaporative air drying at a temperature higher than ambient.

21. The composition of claim 10 wherein X is an N-hydroxysuccinimidyl (NHS) ester.

22. A method for forming particles of a target substance which comprises a composition selected from the group consisting of a polyalkylene glycol (PAG) and a derivative and a conjugate thereof, which method comprises carrying out a GAS process on a solution or suspension of the target substance in a fluid vehicle and using as the anti-solvent fluid a compressed fluid which at the point of its contact with the target substance in the fluid is at a temperature of 25° C. (298 K) or below.

23. A method for forming particles of a target substance selected from the group consisting of one or more polyalkylene glycols (PAGs) and derivatives and conjugates thereof, which method comprises carrying out a GAS process on a solution or suspension of the target substance in a fluid vehicle.

24. The method of claim 22, wherein the anti-solvent fluid used in the GAS process is a compressed fluid which at the point of its contact with the target substance in the fluid vehicle is at a temperature of 25° C. (298 K) or below.

25. The method of claim 22, wherein the GAS process is a Nektar™ SCF particle formation process in which a compressed fluid anti-solvent is used simultaneously both to extract a fluid vehicle from, and to disperse, a solution or suspension containing the target substance, or a precursor thereof, in the vehicle.

26. The method of claim 22, wherein the composition is selected from the group consisting of polyethylene glycol (PEG), polypropylene glycol (PPG), and copolymers derivatives, conjugates and mixtures thereof

27. The method of claim 23, wherein the target substance is selected from the group consisting of PEG, PEG derivatives, PEG conjugates, and mixtures thereof.

28. The method of claim 22, wherein the PAG derivative is an activated PAG containing one or more reactive functional groups X on the main polymer chain(s) and/or at its terminae, where X is an activating group which facilitates subsequent conjugation of the polymer to another substance.

29. The method of claims 22, wherein the PAG conjugate is an activated PAG, covalently bound to at least one active substance Z.

30. The method of claim 22, wherein the composition has a molecular weight of from 2.5 to 40 kDaltons.

31. The method of claim 22, wherein the composition has a melting point of 55° C. or lower.

32. The method of claim 22, wherein the anti-solvent fluid is liquid carbon dioxide.

33. The method of claim 22, wherein the anti-solvent fluid, at the point of its contact with the target substance in the fluid vehicle, is at a pressure above its critical pressure P_{c}.

34. The method of claim 22, wherein the anti-solvent fluid, at the point of its contact with the target substance in the fluid vehicle, is at a pressure of from 75 to 125 bar.

35. The method of claim 22, wherein the temperature at the point where the target substance in the fluid vehicle contacts the anti-solvent is less than 10° C.

36. The method of claim 35, wherein the temperature is less than 5° C.

37. The method of claim 22, which results in a reduction of the particle size of the target substance to 70% or less of that of the starting material.

38. The method of claim 22, which is a single step process and does not involve evaporative air drying of the particles, at a temperature higher than ambient, following their formation, and which yields particles having a residual solvent content of 1000 ppm or less.

39. The method of claim 38, which yields particles having a residual solvent content of 150 ppm or less.

40. A particulate product formed using the method of claim 22.

41. A pharmaceutical composition comprising the particulate product of claim 1 and a pharmaceutically acceptable excipient.
